(19) United States
(12) Patent Application Publication
(10) Pub. No.: US 2026/0325514 A1
(43) Pub. Date: Oct. 22, 2026

(54) METHODS AND COMPOSITIONS FOR TREATING MOVEMENT DISORDERS

(71) Applicant: Edgewise Therapeutics, Inc., Boulder, CO (US)

(72) Inventors: Alan Russell, Boulder, CO (US); Badreddin Edris, San Jose, CA (US)

(21) Appl. No.: 19/396,215

(22) Filed: Nov. 20, 2025

Related U.S. Application Data
(63) Continuation of application No. 17/834,222, filed on Jun. 7, 2022, now abandoned.
Application No. 17/834,222 is a division of application No. 16/998,938, filed on Aug. 20, 2020, now abandoned.
Application No. 16/998,938 is a continuation of application No. PCT/US2019/018626, filed on Feb. 19, 2019.
(60) Provisional application No. 62/632,957, filed on Feb. 20, 2018.
(60) Provisional application No. 62/756,513, filed on Nov. 6, 2018.

(51) Int. Cl. A61K 31/50; A61P 21/00

(52) U.S. Cl. CPC A61K 31/50; A61P 21/00

(57) ABSTRACT

Disclosed herein are methods and compositions for the treatment of movement disorders including neuromuscular disorders, muscular injuries, and spasticity-associated conditions. Methods of treatment include reducing skeletal muscle contractions to reduce muscle damage by inhibiting skeletal muscle myosin II.

### CROSS-REFERENCE TO RELATED APPLICATIONS

This application is a continuation application of U.S. patent application Ser. No. 17/834,222, filed Jun. 7, 2022, which is a divisional application U.S. patent application Ser. No. 16/998,938, filed Aug. 20, 2020, which is a continuation application of International Patent Application No. PCT/US2019/018626, filed Feb. 19, 2019, which claims the benefit of U.S. Provisional Application Ser. No. 62/632,957, filed Feb. 20, 2018, and U.S. Provisional Application Ser. No. 62/756,513, filed Nov. 6, 2018, which applications are incorporated herein by reference.

### BACKGROUND

Skeletal muscle is the largest organ system in the human body, serving two primary purposes. The first is force production to enable muscle contraction, locomotion, and postural maintenance; the second is glucose, fatty acid and amino acid metabolism. The contraction of skeletal muscle during every-day activity and exercise is naturally connected to muscle stress, breakdown and remodeling which is important for muscle adaptation. In individuals with neuromuscular conditions, such as Duchenne Muscular Dystrophy (DMD), muscle contractions lead to continued rounds of amplified muscle breakdown that the body struggles to repair. Eventually, as patients age, a pathophysiological process emerges that leads to excess inflammation, fibrosis, and fatty deposit accumulation in the muscle, portending a steep decline in physical function and contribution to mortality.

DMD is a genetic disorder affecting skeletal muscle and is characterized by progressive muscle degeneration and weakness. There remains a need for treatments that reduce muscle breakdown in patients with neuromuscular conditions such as DMD.

### SUMMARY

In some aspects, methods of treating a neuromuscular condition are described herein. The methods may comprise administering to a subject in need thereof an inhibitor of skeletal muscle contraction. An inhibitor of skeletal muscle contraction may be administered in an amount less than the amount needed to reduce skeletal muscle contraction by 90% relative to a pre-treatment skeletal muscle contraction capacity of said subject.

In some aspects, methods of treating a neuromuscular condition may comprise administering to a subject in need thereof an inhibitor of skeletal muscle contraction. An inhibitor of skeletal muscle contraction may be administered in an amount that reduces skeletal muscle contraction by 5% to 75% relative to a pre-treatment skeletal muscle contraction capacity of said subject.

In some aspects, said inhibitor of skeletal muscle contraction may be administered in an amount that modulates creatinine kinase by 5 to 90% relative to a pre-treatment creatinine kinase level of said subject.

In some aspects, said inhibitor of skeletal muscle contraction may be administered in an amount that modulates an inflammatory marker. The inflammatory marker may be selected from a group consisting of IL-1, IL-6 and TNF-α or conditions that can be measured using magnetic resonance imaging such as edema by 5 to 90% relative to a pre-treatment value of said subject.

In some aspects, said inhibitor of skeletal muscle contraction reduces skeletal muscle contraction by 5% to 90% in an ex vivo assay. In said ex vivo assay, (a) extensor digitorum longus muscle dissected from a mdx mouse may be mounted on an electromagnetic puller and said muscle may be bathed in an oxygenated Krebs solution to maintain muscle function; (b) a test compound may be applied to said muscle; (c) an isometric contraction step may be performed wherein said muscle may be stimulated with a series of five to six electrical pulses; (d) an eccentric contraction step may be performed wherein said muscle may be electrically stimulated at 80-125 Hz for 0.35-0.7 seconds and stretched to 10% to 20% greater than its rested length electrically stimulated at 80-125 Hz for 0.35-0.7 seconds and following each pulse, the force generated by the muscle contraction may be measured; (e) the change in force generated by the muscle contraction from said first pulse to said fifth to sixth pulse in step (d) may be calculated as the test force drop and compared to the change in force generated by the muscle contraction from the first pulse to the sixth pulse in a control sample without exposure to the test compound (control force drop). Muscle membrane damage may also be measured by incubating muscles in procion orange after the isometric or eccentric contraction. Procion orange is a fluorescent dye that is taken up by muscle fibers with injured membranes. The number or proportion of dye-positive fibers is then quantified by histology. When the test force drop and/or proportion of dye-positive fibers may be at least 20% less than the control force drop and/or dye uptake, the test compound may be selected as an inhibitor of skeletal muscle contraction.

In some aspects, said inhibitor of skeletal muscle contraction inhibits ATPase activity in an assay. A myosin S1 fragment may be incubated with polymerized actin in a control and test vessel. A test compound and MgATP may be added to the mixture in the test vessel and MgATP may be added to the control vessel. The amount of ATP consumption over a defined time period in the test vessel may be compared to the amount of ATP consumption in said control vessel. The defined period of time may be 5 minutes to 20 minutes. The ATP consumption may be correlated to the production of NAD+. In some cases, wherein ATP consumption is decreased by at least 20% in said test vessel as compared to said control vessel, said test compound may be selected as an inhibitor of skeletal muscle contraction.

In some aspects, methods of treating a neuromuscular condition may comprise measuring cardiac muscle contraction or force from said cardiac muscle contraction of a subject. An inhibitor of skeletal muscle contraction may be administered to a subject in need thereof. Cardiac muscle contraction or force from said cardiac muscle contraction of said subject may be measured following administration of said inhibitor of skeletal muscle contraction. Cardiac muscle contraction in said subject may be within 10% of said cardiac muscle contraction relative to a pre-treatment capacity.

In some embodiments, said neuromuscular conditions may be selected from Duchenne Muscular Dystrophy, Becker muscular dystrophy, myotonic dystrophy 1, myotonic dystrophy 2, facioscapulohumeral muscular dystrophy, oculopharyngeal muscular dystrophy, limb girdle muscular dystrophy, tendinitis, carpal tunnel syndrome.

In some embodiments, said inhibitor of muscle contraction may be selected from an inhibitor of myosin. In some embodiments, said inhibitor of myosin may be an inhibitor of skeletal muscle myosin II.

In some aspects, methods of treating a movement disorder may comprise administering to a subject in need thereof an inhibitor of skeletal muscle myosin II. In some embodiments, said movement disorder comprises muscle spasticity. In some embodiments, said muscle spasticity may be selected from spasticity associated with multiple sclerosis, Parkinson's disease, Alzheimer's disease, or cerebral palsy, or injury, or a traumatic event such as stroke, traumatic brain injury, spinal cord injury, hypoxia, meningitis, encephalitis, phenylketonuria, or amyotrophic lateral sclerosis.

In some embodiments, said inhibitor of skeletal muscle myosin II may be administered in an amount sufficient to reduce involuntary muscle contractions by 90%. In some embodiments, said inhibitor of skeletal muscle myosin II may be administered in an amount sufficient to reduce involuntary muscle contractions by 25-75%.

In some embodiments, said inhibitor of skeletal muscle myosin II may not impact activities of daily living (ADL) or habitual physical activity. In some embodiments, said inhibitor of skeletal muscle contraction may not impact activities of daily living (ADL) or habitual physical activity.

In some embodiments, said methods further comprise measuring skeletal muscle contraction or force from said skeletal muscle contraction of said subject prior to and following administration of said skeletal muscle myosin II inhibitor to said subject.

In some embodiments, said skeletal muscle contraction of said subject prior to the administering may be within 20% of said skeletal muscle contraction following said administering to said subject. In some embodiments, said skeletal muscle contraction of said subject prior to the administering may be within 10% of said muscle contraction following said administering to said subject.

In some embodiments, said inhibitor of skeletal muscle myosin II may not appreciably inhibit cardiac muscle contraction or force from said cardiac muscle contraction of said subject. In some embodiments, said inhibitor of skeletal muscle myosin II may not appreciably inhibit tidal volume in the lung of said subject.

In some embodiments, said methods further comprise measuring cardiac muscle contraction or force from said cardiac muscle contraction of said subject prior to and following administration of said skeletal muscle myosin II inhibitor. In some cases, said cardiac muscle contraction of said subject prior to the administering may be within 10% of said cardiac muscle contraction following said administering to said subject.

In some embodiments, said contraction-induced injury in skeletal muscle fiber may be from involuntary skeletal muscle contraction. In some embodiments, said involuntary skeletal muscle contraction may be associated with a neuromuscular condition or spasticity-associated condition. In some embodiments, said neuromuscular condition may be Duchenne Muscular Dystrophy.

In some embodiments, said contraction-induced injury in skeletal muscle fiber may be from voluntary skeletal muscle contraction.

In some embodiments, said methods further comprise measuring cardiac muscle contraction or force from said cardiac muscle contraction of said subject prior to and following administration of said skeletal muscle myosin II inhibitor. In some embodiments, said inhibitor of skeletal muscle myosin II may not appreciably inhibit smooth muscle contraction.

In some embodiments, said methods further comprise measuring smooth muscle contraction or force from said smooth muscle contraction of said subject prior to and following administration of said skeletal muscle myosin II inhibitor. In some embodiments, said smooth muscle contraction of said subject prior to the administration may be within 10% of said smooth muscle contraction following said administering.

In some embodiments, said inhibitor of skeletal muscle myosin II inhibits ATPase activity but may not inhibit cardiac muscle myosin S1 ATPase in vitro assays. In some embodiments, said inhibitor of skeletal muscle myosin II may be a sulfonamide, a hydroxycoumarin, a pyridazinone, or a pyrrolidinone.

In some embodiments, said inhibitor of skeletal muscle myosin II may be a sulfonamide. In some embodiments, said inhibitor of skeletal muscle myosin II may be an optionally substituted N-benzyl-p-tolyl-sulfonamide. In some embodiments, the inhibitor of skeletal muscle myosin II is a pyridazinone.

In some embodiments, said skeletal muscle contraction may be measured by an isolated limb assay, grip strength or a leg press assay or a heart rate monitor or an activity monitor. In some embodiments, said administration of the inhibitor of skeletal muscle contraction may not appreciably inhibit release of cardiac troponin or slow skeletal troponin.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1: Comparison between normal and DMD muscle upon exposure to increasing concentrations of calcium.

FIG. 2: Comparison between control and BTS treated muscles in embryos of DMD zebrafish models.

### DETAILED DESCRIPTION

In certain aspects, the disclosure provides methods for treating neuromuscular conditions through selective inhibition of fast-fiber skeletal muscle myosin. In particular, methods of the disclosure may be used in the treatment of DMD and other neuromuscular conditions.

Skeletal muscle is mainly composed of two types of fibers, slow-twitch muscle fiber (i.e., type I) and fast-twitch muscle fiber (i.e., type II). In each muscle, the two types of fibers are configured in a mosaic-like arrangement, with differences in fiber type composition in different muscles and at different points in growth and development. Slow-twitch muscle fibers have excellent aerobic energy production ability. Contraction rate of the slow-twitch muscle fiber is low but tolerance to fatigue is high. Slow-twitch muscle fibers typically have a higher concentration of mitochondria and myoglobin than do fast-twitch fibers and are surrounded by more capillaries than are fast-twitch fibers. Slow-twitch fibers contract at a slower rate due to lower myosin ATPase activity and produce less power compared to fast-twitch fibers, but they are able to maintain contractile function over longer-terms, such as in stabilization, postural control, and endurance exercises.

Fast twitch muscle fibers in humans are further divided into two main fiber types depending on the specific fast skeletal myosin they express (Type IIa, IIx/d). A third type of fast fiber (Type IIb) exists in other mammals but is rarely identified in human muscle. Fast-twitch muscle fibers have excellent anaerobic energy production ability and are able to generate high amounts of tension over a short period of time. Typically, fast-twitch muscle fibers have lower concentrations of mitochondria, myoglobin, and capillaries compared to slow-twitch fibers, and thus can fatigue more quickly. Fast-twitch muscles produce quicker force required for power and resistance activities.

Proportion of the type I and type II can vary in different individuals. For example, non-athletic individuals can have close to 50% of each muscle fiber type. Power athletes can have a higher ratio of fast-twitch fibers, e.g., 70-75% type II in sprinters. Endurance athletes can have a higher ratio of slow-twitch fibers, e.g., 70-80% in distance runners. The proportion of the type I and type II fibers can also vary depending on the age of an individual. The proportion of type II fibers, especially the type IIx, can decline as an individual ages, resulting in a loss in lean muscle mass.

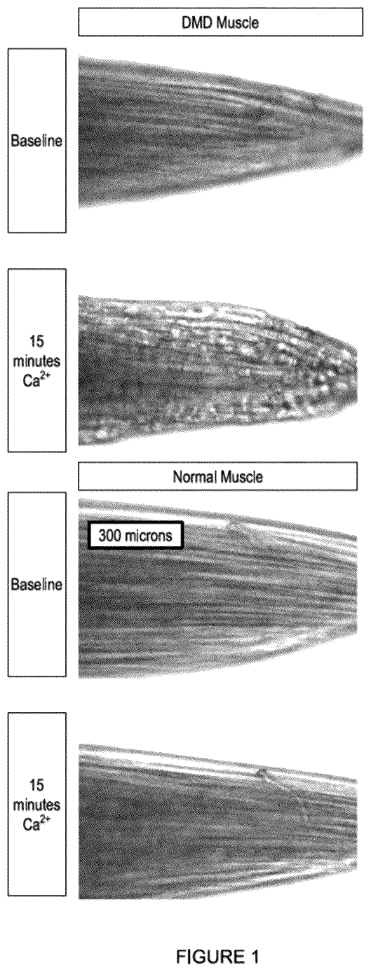

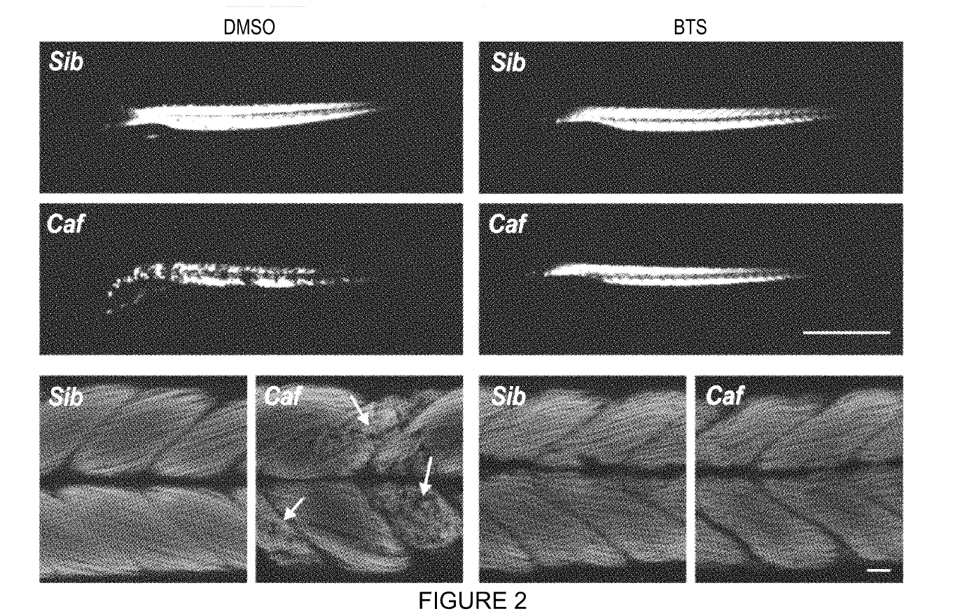

The contractile action of skeletal muscle leads to muscle damage in subjects with neuromuscular disease, e.g., DMD, and this damage appears to be more prevalent in fast fibers. It has been noted that acute force drop after lengthening injury is greater in predominantly fast type II fiber muscles (ie EDL) compared to predominantly slow type I fiber muscles (ie soleus) in dystrophy mouse models. It has also been demonstrated that the degree of acute force drop and histological damage in dystrophy mouse models is proportional to peak force development during lengthening injury. Excessive contraction-induced injuries, which precede the inflammation and irreversible fibrosis that characterizes late-stage DMD pathology are shown in FIG. 1 [Figure adapted: Claflin and Brooks, Am J Brooks, Physiol Cell, 2008,]. Contraction induced muscle damage in these patients may be reduced by limiting peak force generation in type II fibers and possibly increasing reliance on healthier type I fibers. N-benzyl-p-tolyl-sulfonamide (BTS), an inhibitor of fast-fiber skeletal muscle myosin, has been shown to protect muscles from pathological muscle derangement in embryos from zebrafish model of DMD as shown in FIG. 2. [Source: Li and Arner, PLoSONE, 2015].

Inhibitors of skeletal muscle myosin that are not selective for the type II fibers may lead to excessive inhibition of skeletal muscle contraction including impairing respiratory function and cardiac activity as the heart shares several structural components (such as type I myosin) with type I skeletal muscle fibers. As contractions of type II fibers are believed to drive pathological and irreversible muscle damage, the disclosure provides a selective inhibitor of fast-fiber skeletal muscle myosin as a treatment option for DMD and other neuromuscular conditions. The targeted inhibition of type II skeletal muscle myosin may reduce skeletal muscle contractions while minimizing the impact on a subject's daily activities.

Methods discussed herein may be used for the treatment of neuromuscular conditions and movement disorders. Examples of neuromuscular conditions include but are not limited to Duchenne Muscular Dystrophy, Becker muscular dystrophy, myotonic dystrophy 1, myotonic dystrophy 2, facioscapulohumeral muscular dystrophy, oculopharyngeal muscular dystrophy, limb girdle muscular dystrophies, tendinitis and carpal tunnel syndrome. Examples of movement disorders include but are not limited to muscle spasticity disorders, spasticity associated with multiple sclerosis, Parkinson's disease, Alzheimer's disease, or cerebral palsy. Methods of the disclosure may be used to treat movement disorders from injury or a traumatic event such as stroke, traumatic brain injury, spinal cord injury, hypoxia, meningitis, encephalitis, phenylketonuria, or amyotrophic lateral sclerosis. Also included are other conditions that may respond to the inhibition of skeletal myosin II, skeletal troponin C, skeletal troponin I, skeletal tropomyosin, skeletal troponin T, skeletal regulatory light chains, skeletal myosin binding protein C or skeletal actin.

Presented herein are methods to treat neuromuscular and movement disorders by reduction of skeletal muscle contraction. Treatment of subjects with neuromuscular and movement disorders with a selective fast skeletal muscle (type II) myosin inhibitor may reduce muscle breakdown by preventing excessive uncoordinated muscle contractures resulting in less muscle damage. Furthermore, methods of the disclosure may reduce muscle damage while minimizing the impact on physical function in subjects. Preservation of function may occur both by limiting damaging levels of force generation in type II fibers and by increasing reliance on healthier type I fibers. Reduction of skeletal muscle contraction or uncoordinated muscle contractures can be reduced by the inhibition of skeletal myosin II. In certain embodiments, the inhibitor of skeletal myosin II is a sulfonamide, a hydroxycoumarin, or a pyrrolidinone. The inhibitor of skeletal muscle myosin II can be an analog of N-benzyl-p-tolyl-sulfonamide (BTS). In certain embodiments, the inhibitor of skeletal muscle myosin II is a pyridazinone. As used herein, a pyridazinone refers to a compound represented by the structure

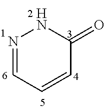

and substituted versions thereof. For example, a pyridazinone may be substituted at one or more positions such as substituted at the 2-, 4-, 5-, or 6-positions of the pyridazinone. In certain embodiments, a pyridazinone is substituted at both the 2-position and the 6-position. Substituents on the pyridazinone may be selected from optionally substituted alkyl groups, optionally substituted carbocycles, e.g., cycloalkyl and aryl rings, and optionally substituted heterocycles, heterocycloalkyl and heteroaryl rings. In certain embodiments, a pyridazinone is selected from a compound or salt thereof described in PCT publication No. WO2016/023877, the contents of which are incorporated herein by reference.

The term “substituted” refers to moieties, e.g., pyridazinone, having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O) R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O) R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O) R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O) R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O) R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O) R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

A subject's activities of daily life (ADL) or habitual physical activity may be monitored prior to and following the treatment with an inhibitor of skeletal muscle contraction. ADL or habitual physical activity is subject-dependent and may range from simple walking to extensive exercise depending on the subject's ability and routine. Treatment options and dosages of the skeletal muscle contraction inhibitors discussed herein may be personalized to a subject such that the ADL and habitual physical activity remains unchanged.

In some aspects, methods of treating neuromuscular conditions or movement disorders may comprise administering to a subject in need thereof an inhibitor of skeletal muscle contraction. An inhibitor of skeletal muscle contraction may be given in an amount relative to the amount needed to reduce skeletal muscle contraction by 50%. The inhibitor of skeletal muscle contraction may be administered in an amount less than the amount needed to reduce skeletal muscle contraction by 50% relative to a pre-treatment skeletal muscle contraction capacity of the subject. The inhibitor of skeletal muscle contraction may be administered in an amount that reduces skeletal muscle contraction by 5% to 45% relative to a pre-treatment skeletal muscle contraction capacity of said subject. In some cases, the inhibitor may be administered in an amount that reduces skeletal muscle contraction by less than 10%, less than 15%, less than 20%, less than 25%, less than 30%, less than 35%, less than 40%, less than 45% or even less than 50% relative to a pre-treatment skeletal muscle contraction capacity of said subject. In certain embodiments, the inhibitor may be administered in an amount that reduces skeletal muscle contraction from 1% to 50% relative to a pre-treatment skeletal muscle contraction capacity of said subject.

In some aspects, methods of treating neuromuscular conditions or movement disorders may comprise administering to a subject in need thereof an inhibitor of type I skeletal muscle contraction. An inhibitor of type I skeletal muscle contraction may be given in an amount relative to the amount needed to reduce type I skeletal muscle contraction by 20%. The inhibitor of type I skeletal muscle contraction may be administered in an amount less than the amount needed to reduce type I skeletal muscle contraction by 20% relative to a pre-treatment type I skeletal muscle contraction capacity of the subject. The inhibitor of type I skeletal muscle contraction may be administered in an amount that reduces type I skeletal muscle contraction by 0.01% to 20%, such as 1% to 15%, such as 1% to 10%, relative to a pre-treatment type I skeletal muscle contraction capacity of said subject. In some cases, the inhibitor may be administered in an amount that reduces type I skeletal muscle contraction by less than 0.01%, less than 0.1%, less than 0.5%, less than 1%, less than 5%, less than 10%, less than 15% or less than 20% relative to a pre-treatment type I skeletal muscle contraction capacity of said subject. In certain embodiments, the inhibitor may be administered in an amount that reduces type I skeletal muscle contraction from 0.01% to 20% relative to a pre-treatment type I skeletal muscle contraction capacity of said subject.

In some aspects, methods of treating neuromuscular conditions or movement disorders may comprise administering to a subject in need thereof an inhibitor of type II skeletal muscle contraction. An inhibitor of type II skeletal muscle contraction may be given in an amount relative to the amount needed to reduce type II skeletal muscle contraction by 90%. The inhibitor of type II skeletal muscle contraction may be administered in an amount less than the amount needed to reduce type II skeletal muscle contraction by 90% relative to a pre-treatment type II skeletal muscle contraction capacity of the subject. The inhibitor of type II skeletal muscle contraction may be administered in an amount that reduces type II skeletal muscle contraction by 5% to 90%, such as 5% to 80%, such as 5% to 75%, such as 5% to 70% relative to a pre-treatment type II skeletal muscle contraction capacity of said subject. In some cases, the inhibitor may be administered in an amount that reduces type II skeletal muscle contraction by 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more or even 90% or more relative to a pre-treatment type II skeletal muscle contraction capacity of said subject. In certain embodiments, the inhibitor may be administered in an amount that reduces type II skeletal muscle contraction by from 1% to 50% relative to a pre-treatment type II skeletal muscle contraction capacity of said subject.

In some aspects, methods of treating contraction-induced injury in skeletal muscle fiber may comprise administering to a subject in need thereof an inhibitor of skeletal muscle contraction and/or skeletal muscle myosin II. In certain embodiments, the inhibitor does not appreciably inhibit cardiac muscle contraction.

In certain embodiments, the contraction-induced injury in skeletal muscle fiber is from involuntary skeletal muscle contraction. The involuntary skeletal muscle contraction may be associated with a neuromuscular condition or spasticity-associated condition. In certain embodiments, the contraction-induced injury in skeletal muscle fiber may be from voluntary skeletal muscle contraction, e.g., physical exercise.

In certain embodiments, the administration of the inhibitor of skeletal muscle contraction to a subject modulates one or more biomarkers associated with muscle contraction. Examples of biomarkers include but are not limited to creatinine kinase (CK), Troponin T (TnT), Troponin C (TnC), Troponin I (TnI), pyruvate kinase (PK), lactate dehydrogenase (LDH), myoglobin, isoforms of TnI (such as cardiac, slow skeletal, fast skeletal muscles) and inflammatory markers (IL1, IL6, IL4, TNF-α). Biomarkers may also include measures of muscle inflammation for example, edema. The level of biomarkers described herein may increase after the administration of the inhibitor relative to a pre-treatment level of the biomarkers. Alternatively, the level of biomarkers may decrease after the administration of the inhibitor relative to a pre-treatment level of the biomarkers. The modulation of one or more biomarkers with an inhibitor described herein may indicate treatment of a neuromuscular condition such as those described herein.

Levels of CK in a subject increase when the subject is active as compared to when the subject is inactive (e.g., sleeping) and therefore CK is a potential metric for evaluating skeletal muscle breakdown caused by skeletal muscle contraction. In certain embodiments, an inhibitor of skeletal muscle contraction may be administered to a subject prior to mild, moderate or strenuous activity to reduce or prevent skeletal muscle breakdown from the activity. Moderate to strenuous activity may be dependent on a subject's abilities and may include physical exercise that increases the heart rate by at least 20% or more, such as about 50% or more relative to the subject's resting heart rate. Examples of moderate to strenuous activity include walking, running, weight lifting, biking, swimming, hiking, etc.

In certain embodiments, the inhibitor of skeletal muscle contraction is administered prior to, during, or after moderate or strenuous activity to reduce or prevent skeletal muscle breakdown from the activity. The inhibitor of skeletal muscle contraction may reduce the subject's level of CK relative to the untreated subject performing the same activity. The level of CK may be measured in the peripheral blood of the subject during or after the activity. The administration of an inhibitor described herein may reduce the level of CK by 5% to 90%, such as 5% to 80%, such as 10% to 75%, in an active subject relative to the untreated subject performing the same activity, thereby reducing or preventing skeletal muscle breakdown from the activity. The administration of an inhibitor described herein may modulate the level of CK by about 5% to about 90% relative to the untreated subject performing the same activity, thereby reducing or preventing skeletal muscle breakdown from the activity. The administration of an inhibitor described herein may reduce the level of CK by at least about 5% relative to the untreated subject performing the same activity thereby reducing or preventing skeletal muscle breakdown from the activity. The administration of an inhibitor described herein may modulate the level of CK by 90% or less relative to the untreated subject performing the same activity. The administration of an inhibitor described herein may reduce the level of CK by about 5% to about 15%, about 5% to about 25%, about 5% to about 35%, about 5% to about 45%, about 5% to about 55%, about 5% to about 65%, about 5% to about 75%, about 5% to about 85%, about 5% to about 90%, about 15% to about 25%, about 15% to about 35%, about 15% to about 45%, about 15% to about 55%, about 15% to about 65%, about 15% to about 75%, about 15% to about 85%, about 15% to about 90%, about 25% to about 35%, about 25% to about 45%, about 25% to about 55%, about 25% to about 65%, about 25% to about 75%, about 25% to about 85%, about 25% to about 90%, about 35% to about 45%, about 35% to about 55%, about 35% to about 65%, about 35% to about 75%, about 35% to about 85%, about 35% to about 90%, about 45% to about 55%, about 45% to about 65%, about 45% to about 75%, about 45% to about 85%, about 45% to about 90%, about 55% to about 65%, about 55% to about 75%, about 55% to about 85%, about 55% to about 90%, about 65% to about 75%, about 65% to about 85%, about 65% to about 90%, about 75% to about 85%, about 75% to about 90%, or about 85% to about 90% relative to the untreated subject performing the same activity, thereby reducing or preventing skeletal muscle breakdown from the activity. The administration of an inhibitor described herein may modulate the level of CK by about 5%, about 15%, about 25%, about 35%, about 45%, about 55%, about 65%, about 75%, about 85%, or about 90% relative to the untreated subject performing the same activity, thereby reducing or preventing skeletal muscle breakdown from the activity.

The administration of the inhibitor of skeletal muscle contraction to a subject may modulate the levels of inflammatory markers, e.g., reduce the level of one or more inflammatory markers relative to the untreated subject or the subject prior to treatment. The level of inflammatory markers may be measured in the peripheral blood of the subject. Examples of inflammatory markers may include but are not limited to IL-1, IL-6 and TNF-α. Inflammatory markers may also be in the form of conditions such as edema which may be measured using magnetic resonance imaging. The level of inflammatory markers in the peripheral blood may increase after the administration of the inhibitor relative to a pre-treatment level of inflammatory marker for the subject. Alternatively, the level of inflammatory markers in the peripheral blood may decrease after the administration of the inhibitor relative to a pre-treatment level of inflammatory marker for the subject. The administration of an inhibitor described herein may modulate the level of inflammatory markers by 5% to 90% relative to a pre-treatment level of inflammatory marker for the subject. In some cases, the level of inflammatory markers may be modulated by about 5% to about 90% relative to a pre-treatment level of inflammatory markers of the subject. In some cases, the level of inflammatory markers may be modulated by at least about 5% relative to a pre-treatment level of inflammatory markers of the subject. In some cases, the level of inflammatory markers may be modulated by at most about 90% relative to a pre-treatment level of inflammatory markers of the subject. In some cases, the level of inflammatory markers may be modulated by about 5% to about 15%, about 5% to about 25%, about 5% to about 35%, about 5% to about 45%, about 5% to about 55%, about 5% to about 65%, about 5% to about 75%, about 5% to about 85%, about 5% to about 90%, about 15% to about 25%, about 15% to about 35%, about 15% to about 45%, about 15% to about 55%, about 15% to about 65%, about 15% to about 75%, about 15% to about 85%, about 15% to about 90%, about 25% to about 35%, about 25% to about 45%, about 25% to about 55%, about 25% to about 65%, about 25% to about 75%, about 25% to about 85%, about 25% to about 90%, about 35% to about 45%, about 35% to about 55%, about 35% to about 65%, about 35% to about 75%, about 35% to about 85%, about 35% to about 90%, about 45% to about 55%, about 45% to about 65%, about 45% to about 75%, about 45% to about 85%, about 45% to about 90%, about 55% to about 65%, about 55% to about 75%, about 55% to about 85%, about 55% to about 90%, about 65% to about 75%, about 65% to about 85%, about 65% to about 90%, about 75% to about 85%, about 75% to about 90%, or about 85% to about 90% relative to a pre-treatment level of inflammatory markers of the subject. In some cases, the level of inflammatory markers may be modulated by about 5%, about 15%, about 25%, about 35%, about 45%, about 55%, about 65%, about 75%, about 85%, or about 90% relative to a pre-treatment level of inflammatory markers of the subject.

The administration of the inhibitor of skeletal muscle contraction to a subject may modulate the levels of circulating fast skeletal muscle Troponin I (fS-TnI). The level of fS-TnI may be measured in the peripheral blood. The level of fS-TnI in the peripheral blood may increase after the administration of the inhibitor relative to a pre-treatment level of fS-TnI for the subject. Alternatively, the level of fS-TnI in the peripheral blood may decrease after the administration of the inhibitor relative to a pre-treatment level of fS-TnI for the subject. The administration of an inhibitor described herein may modulate the level of fS-TnI by 5% to 90% relative to a pre-treatment level of fS-TnI for the subject. In some cases, the level of fS-TnI may be modulated by at least about 5% relative to a pre-treatment level of fS-TnI of the subject. In some cases, the level of fS-TnI may be modulated by at most about 90% relative to a pre-treatment level of fS-TnI of the subject. In some cases, the level of fS-TnI may be modulated by about 5% to about 15%, about 5% to about 25%, about 5% to about 35%, about 5% to about 45%, about 5% to about 55%, about 5% to about 65%, about 5% to about 75%, about 5% to about 85%, about 5% to about 90%, about 15% to about 25%, about 15% to about 35%, about 15% to about 45%, about 15% to about 55%, about 15% to about 65%, about 15% to about 75%, about 15% to about 85%, about 15% to about 90%, about 25% to about 35%, about 25% to about 45%, about 25% to about 55%, about 25% to about 65%, about 25% to about 75%, about 25% to about 85%, about 25% to about 90%, about 35% to about 45%, about 35% to about 55%, about 35% to about 65%, about 35% to about 75%, about 35% to about 85%, about 35% to about 90%, about 45% to about 55%, about 45% to about 65%, about 45% to about 75%, about 45% to about 85%, about 45% to about 90%, about 55% to about 65%, about 55% to about 75%, about 55% to about 85%, about 55% to about 90%, about 65% to about 75%, about 65% to about 85%, about 65% to about 90%, about 75% to about 85%, about 75% to about 90%, or about 85% to about 90% relative to a pre-treatment level of fS-TnI of the subject. In some cases, the level of fS-TnI may be modulated by about 5%, about 15%, about 25%, about 35%, about 45%, about 55%, about 65%, about 75%, about 85%, or about 90% relative to a pre-treatment level of fS-TnI of the subject.

Isoforms of troponin may be measured in a subject prior to and following the administration of a skeletal muscle contraction inhibitor. Inhibition of skeletal muscle contraction may not inhibit some isoforms of troponin, such as cardiac troponin I (cTnI) or slow skeletal troponin I (ssTnI). In some cases, the inhibition of skeletal muscle contraction may not appreciably inhibit cTnI or ssTnI. As used herein with regard to cTnI or ssTnI, the phrase not appreciably refers to the cTnI or ssTnI reduced by less than 10%, less than 8%, less than 6%, less than 4%, less than 2%, less than 1%, less than 0.5% or even less than 0.1% relative to the cTnI or ssTnI prior to the administration of the inhibitor.

The administration of the inhibitor of skeletal muscle contraction may reduce involuntary muscle contractions. Involuntary muscle contractions may be reduced by 20% to 90% relative to involuntary muscle contractions prior to the administration of the inhibitor. In some cases, involuntary muscle contractions may be reduced by at least about 20% relative to pre-treatment involuntary muscle contractions. In some cases, involuntary muscle contractions may be reduced by at most about 90% relative to pre-treatment involuntary muscle contractions. In some cases, involuntary muscle contractions may be reduced by about 20% to about 25%, about 20% to about 30%, about 20% to about 40%, about 20% to about 50%, about 20% to about 70%, about 20% to about 75%, about 20% to about 80%, about 20% to about 85%, about 20% to about 90%, about 25% to about 30%, about 25% to about 40%, about 25% to about 50%, about 25% to about 70%, about 25% to about 75%, about 25% to about 80%, about 25% to about 85%, about 25% to about 90%, about 30% to about 40%, about 30% to about 50%, about 30% to about 70%, about 30% to about 75%, about 30% to about 80%, about 30% to about 85%, about 30% to about 90%, about 40% to about 50%, about 40% to about 70%, about 40% to about 75%, about 40% to about 80%, about 40% to about 85%, about 40% to about 90%, about 50% to about 70%, about 50% to about 75%, about 50% to about 80%, about 50% to about 85%, about 50% to about 90%, about 70% to about 75%, about 70% to about 80%, about 70% to about 85%, about 70% to about 90%, about 75% to about 80%, about 75% to about 85%, about 75% to about 90%, about 80% to about 85%, about 80% to about 90%, or about 85% to about 90% relative to pre-treatment involuntary muscle contractions. In some cases, involuntary muscle contractions may be reduced by about 20%, about 25%, about 30%, about 40%, about 50%, about 70%, about 75%, about 80%, about 85%, or about 90% relative to pre-treatment involuntary muscle contractions.

The inhibitor of skeletal muscle contraction may be used to improve activities of daily living (ADL) or habitual physical activity in a subject as mature, functional undamaged muscle may be restored. Examples of ADL or habitual activities include but are not limited to stair climb, time to get up, timed chair rise, habitual walk speed, North Star Ambulatory assessment, incremental/endurance shuttle walk and 6 minute walk distance tests. ADL or habitual physical activity levels or capacity may be measured prior to and following the administration of a skeletal muscle inhibitor. Inhibition of skeletal muscle contraction may not affect ADL or habitual physical activity. In some cases, the inhibition of skeletal muscle contraction may not appreciably affect ADL or habitual physical activity. As used herein with regard to ADL or habitual physical activity, the phrase not appreciably refers to the level of ADL or habitual activity reduced by less than 20%, less than 15%, less than 10%, less than 8%, less than 6%, less than 4%, less than 2%, less than 1%, less than 0.5% or even less than 0.1% relative to the ADL or habitual activity prior to the administration of the inhibitor. Skeletal muscle contraction or force in a subject may be measured prior to and following the administration of the inhibitor of skeletal muscle contraction. Such measurements may be performed to generate a dose response curve for the inhibitor of skeletal muscle contraction. Dosage of the inhibitor of skeletal muscle contraction may be adjusted by about 5% to 50% relative to a dose that reduces type II skeletal muscle contraction by 90%. In some cases, dosage of the skeletal muscle contraction inhibitor may be adjusted by at least about 5% relative to a dose that reduces type II skeletal muscle contraction by 90%. In some cases, dosage of the skeletal muscle contraction inhibitor may be adjusted by at most about 50% relative to a dose that reduces type II skeletal muscle contraction by 90%. In some cases, dosage of the skeletal muscle contraction inhibitor may be adjusted by about 5% to about 10%, about 5% to about 15%, about 5% to about 20%, about 5% to about 25%, about 5% to about 30%, about 5% to about 35%, about 5% to about 40%, about 5% to about 50%, about 10% to about 15%, about 10% to about 20%, about 10% to about 25%, about 10% to about 30%, about 10% to about 35%, about 10% to about 40%, about 10% to about 50%, about 15% to about 20%, about 15% to about 25%, about 15% to about 30%, about 15% to about 35%, about 15% to about 40%, about 15% to about 50%, about 20% to about 25%, about 20% to about 30%, about 20% to about 35%, about 20% to about 40%, about 20% to about 50%, about 25% to about 30%, about 25% to about 35%, about 25% to about 40%, about 25% to about 50%, about 30% to about 35%, about 30% to about 40%, about 30% to about 50%, about 35% to about 40%, about 35% to about 50%, or about 40% to about 50% relative to a dose that reduces type II skeletal muscle contraction by 90%. In some cases, dosage of the skeletal muscle contraction inhibitor may be adjusted by about 10%, about 12%, about 15%, about 18%, about 20%, about 25%, about 30%, about 35%, about 40%, about 45% or about 50% relative to a dose that reduces type II skeletal muscle contraction by 90%. Skeletal muscle contraction may be measured by a muscle force test after nerve stimulation using surface electrodes (e.g., foot plantar flexion after peroneal nerve stimulation in the leg), isolated limb assay, heart rate monitor or an activity monitor or equivalents thereof prior to and following the administration of a skeletal muscle contraction inhibitor.

Cardiac muscle force or cardiac muscle contraction of a subject may be measured prior to and following the administration of an inhibitor of skeletal muscle contraction. Inhibition of skeletal muscle contraction may not inhibit cardiac muscle contraction or cardiac muscle force. In some embodiments, the inhibition of skeletal muscle contraction may not appreciably inhibit cardiac muscle contraction. In certain embodiments with regard to cardiac muscle contraction, the phrase not appreciably refers to cardiac muscle force reduced by less than 10%, less than 8%, less than 6%, less than 4%, less than 2%, less than 1%, less than 0.5% or even less than 0.1% relative to the cardiac muscle force prior to the administration of the inhibitor. Cardiac muscle force or cardiac muscle contraction of a subject following the administration of an inhibitor of skeletal muscle contraction may be within 0.1% to 10% of the cardiac muscle contraction or cardiac muscle force prior to the administration of the inhibitor. Cardiac muscle force or cardiac muscle contraction may be measured using an echocardiogram (fractional shortening) or other equivalent tests.

Tidal volume in lung in a subject may be measured prior to and following the administration of a skeletal muscle contraction inhibitor. Inhibition of skeletal muscle contraction may not inhibit tidal volume in a lung. In some cases, the inhibition of skeletal muscle contraction may not appreciably inhibit tidal volume in a lung. In certain embodiments with regard to tidal lung volume in a lung, the phrase not appreciably refers to the tidal volume in a lung reduced by less than 10%, less than 8%, less than 6%, less than 4%, less than 2%, less than 1%, less than 0.5% or less than 0.1% relative to the tidal volume in a lung prior to the administration of the inhibitor. Tidal volume in a lung in a subject may be measured using forced volume in one second test (FEV1) or forced vital capacity test (FVC) or equivalents thereof.

Smooth muscle contraction in a subject may be measured prior to and following the administration of a skeletal muscle contraction inhibitor. Inhibition of skeletal muscle contraction may not inhibit smooth muscle contraction. In some cases, the inhibition of skeletal muscle contraction may not appreciably inhibit smooth muscle contraction. As used herein with regard to smooth muscle contraction, the phrase not appreciably refers to the smooth muscle contraction reduced by less than 10%, less than 8%, less than 6%, less than 4%, less than 2%, less than 1%, less than 0.5% or even less than 0.1% relative to the smooth muscle contraction prior to the administration of the inhibitor. Smooth muscle contraction in a subject may be evaluated by measuring a subject's blood pressure.

Neuromuscular coupling in a subject may be measured prior to and following the administration of a skeletal muscle contraction inhibitor. Inhibition of skeletal muscle contraction, with an inhibitor described herein, may not impair nerve conduction, neurotransmitter release or electrical depolarization of skeletal muscle in a subject. In some cases, the inhibition of skeletal muscle contraction may not appreciably impair neuromuscular coupling in a subject. As used herein with regard to neuromuscular coupling, the phrase not appreciably refers to a level of neuromuscular coupling in the subject reduced by less than 10%, less than 8%, less than 6%, less than 4%, less than 2%, less than 1%, less than 0.5% or less than 0.1% relative to the level of neuromuscular coupling in the subject prior to the administration of the inhibitor. Neuromuscular coupling in a subject may be evaluated by measuring nerve induced electrical depolarization of skeletal muscle by the recording of electrical activity produced by skeletal muscles after electrical or voluntary stimulation with electromyography (EMG) using surface or needle electrodes.

In some aspects, the method of treating a neuromuscular condition or movement disorder can comprise administering to a subject an inhibitor of skeletal muscle contraction wherein the inhibitor of skeletal muscle contraction may inhibit myosin ATPase activity, native skeletal muscle myofibril ATPase (calcium regulated) or a reconstituted S1 with actin, tropomyosin and troponin. In vitro assays may be used to test the effect of the test compound or inhibitor on the myosin ATPase activity. Test compounds can be screened for assessing their inhibitory activity of muscle contraction. Inhibitory activity can be measured using a absorbance assay to determine actin-activated ATPase activity. Rabbit muscle myosin sub-fragment 1 (S1) can be mixed with polymerized actin and distributed into wells of assay plates without nucleotides. Test compounds can then be added into the wells with a pin array. The reaction can be initiated with MgATP. The amount of ATP consumption over a defined time period in the test vessel may be compared to the amount of ATP consumption in a control vessel. The defined period of time may be 5 minutes to 20 minutes. The ATP consumption can be determined by direct or indirect assays. The test compounds that reproducibly and strongly inhibited the myosin S1 ATPase activity can be evaluated further in dose response assay to determine IC50 for the compound ex vivo on dissected muscles. The assay may measure ATPase activity indirectly by coupling the myosin to pyruvate kinase and lactate dehydrogenase to provide an absorbance detection method at 340 nm based upon the conversion of NADH to NAD+ driven by ADP accumulation. In some cases, wherein ATP consumption is decreased by at least 20% in said test vessel than said control vessel, said test compound may be selected as an inhibitor of skeletal muscle contraction. A test compound may be selected when there is at least 20% greater inhibition of NAD+ generation in a kinetic assay.

The inhibitor or test compound selected may not inhibit cardiac muscle myosin S1 ATPase in in vitro assays. In some cases, the cardiac muscle myosin S1 ATPase or cardiac myofibrils or reconstituted system may be inhibited by less than 10%, less than 8%, less than 5%, less than 3%, less than 2%, less than 1% or less than 0.5% when a test compound or inhibitor of skeletal muscle contraction is tested in an in-vitro assay.

Test compounds of skeletal muscle contraction may be tested on skinned fibers. Single skeletal muscle fibers, treated so as to remove membranes and allow for a direct activation of contraction after calcium administration may be used. An inhibitor may inhibit contraction of a single skeletal muscle by about 5% to about 90% relative to a pre-treatment value or an untreated control single skeletal muscle. An inhibitor may inhibit contraction of a single skeletal muscle by at least about 5% relative to a pre-treatment value or an untreated control single skeletal muscle. An inhibitor may inhibit contraction of a single skeletal muscle by at most about 90% relative to a pre-treatment value or an untreated control single skeletal muscle. An inhibitor may inhibit contraction of a single skeletal muscle by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% relative to a pre-treatment capacity or an untreated control single skeletal muscle. An inhibitor may inhibit contraction of a single skeletal muscle by about 5%, about 10%, about 20%, about 30%, about 40%, about 50%, about 60%, about 70%, about 80%, or about 90% relative to a pre-treatment capacity or an untreated control single skeletal muscle.

The effect of a test compound on slow type I skeletal muscle fibers, cardiac muscle bundles or lung muscle fibers, may be evaluated. A test compound or inhibitor may be selected so as not to appreciably modulate the function of slow type I skeletal muscle fibers, cardiac muscle bundles or lung muscle fibers and be specific for type II skeletal muscles. As used herein, the term “appreciably modulate” can refer to the contraction capacity of muscles following the inhibitor administration to be reduced less than 10%, less than 8%, less than 6%, less than 4%, less than 2%, less than 1%, less than 0.5% or even less than 0.1% relative to the muscle force/contraction prior to the administration of the inhibitor.

In some aspects, a method of treating a neuromuscular condition or a movement disorder may comprise administering to a subject in need thereof an inhibitor of skeletal muscle contraction wherein the inhibitor of skeletal muscle contraction reduces skeletal muscle contraction by 5% to 90% in an ex vivo assay. The ex vivo assays used may be mouse models. The mouse models used may be dystrophy mouse models such as an mdx mouse. The mdx mouse has a point mutation in its dystrophin gene, changing the amino acid coding for a glutamine to a threonine producing a nonfunctional dystrophin protein resulting in DMD where there is increased muscle damage and weakness. Extensor digitorum longus muscles may be dissected from mdx mice and mounted on a lever arm. The muscles may be bathed in an oxygenated Krebs solution to maintain muscle function. A test compound or inhibitor of skeletal muscle contraction may be applied to the muscles. An isometric (fixed length) contraction step may then be performed wherein the muscles are stimulated with a series of electrical pulses. An eccentric (lengthening) contraction step may be performed wherein the muscles are stretched to 10%, 15%, 20%, 25%, or 30% greater than its rested length, while relaxed or while stimulated with an electrical pulse. This may be repeated 4, 5, 6, 7 or 8 times to cause muscle fiber injury. The electric pulses may have a frequency of 110 Hz to 150 Hz. The electric pulse may have a frequency of 110, 115, 120, 125, 130, 135, 140, 145 or 150 Hz. A series of electric pulses may comprise of individual pulses of different frequencies. The time period of each pulse in the series of electric pulses may be between 0.1 second to 0.5 seconds for each pulse. The time for each pulse may be 0.1, 0.2, 0.3, 0.35, 0.4 or 0.5 seconds. Muscle membrane damage may also be measured by incubating muscles in procion orange after the isometric or eccentric contraction. Procion orange is a fluorescent dye that is taken up by muscle fibers with injured membranes. The number or proportion of dye-positive fibers may then quantified by histology. When the test force drop and/or proportion of dye-positive fibers may be at least 20% less than the control force drop and/or dye uptake, the test compound may be selected as an inhibitor of skeletal muscle contraction.

Isometric or eccentric set of contractions, the force generated by the muscle may be measured. The change in force generated by the muscle before and after an isometric or eccentric set of contractions may be calculated as the test force drop and compared to the change in force generated by the muscle contraction from the first pulse to the last pulse in a control sample without exposure to the test compound (control force drop). Force drop can be used as a surrogate of muscle injury and a test compound or inhibitor may be selected when the test force drop is at least 20% less than the control force drop.

### EXAMPLES

Efficacy of a test compound can be determined by ex vivo and in vivo assays by comparing muscles from control and dystrophic mice.

### Example 1: Ex Vivo Assay for Assessing Contractile Properties

Muscles can be prepared by dissecting from control (C57BL/10ScSn) and dystrophic (mdx) mice. Muscles comprised mostly of fast-twitch muscle fibers, such as diaphragm strips or intact extensor digitorum longus (EDL) limb muscle, can be used. Muscles can be dissected from young or adult mice, 30- to 110-days-old mice. Muscles can be immersed in physiological solution of 25 mM Hepes buffered to pH 7.4. The physiological solution may contain a fluorescent, low molecular weight dye (0.2% procion orange in Ringer's solution). The physiological solution can be continuously oxygenated and maintained at room temperature or about 23 degrees Celsius. Muscles can be mounted horizontally or vertically in a muscle bath, attached by their bony or tendinous insertions to a fixed post at one end and to the lever of a dual-mode servomotor system at the other. This experimental set up can allow force measurements as well as changes in muscle length by a predetermined velocity and amount. Muscles can be stimulated by using two platinum plate electrodes placed on both sides of the muscle. The muscle can then be adjusted to an optimal length (L₀) that allows maximal twitch force to be achieved. Once L₀ is identified, muscle fiber length can be measured with fine calipers.

A test compound can be applied to control and mdx muscles to assess the contractile properties, especially the muscle strength measured as force generated by the muscles. Untreated or vehicle (DMSO) treated muscles can be used for comparison. Control and mdx muscles can be subjected to one of the following procedures: (a) eccentric contraction regimen comprising five maximal stimulation trains (frequency of 80 Hz for 700-ms duration), with the muscle being lengthened at a velocity of 0.5 L₀/s through a distance of 10% L₀ during the final 200 ms; (b) isometric contraction regimen comprising five maximal stimulation trains, with muscle maintained at L₀ and the force-time integral matched to the eccentric protocol; (c) passive lengthening without muscle stimulation, with the lengthening parameters matched to the eccentric procedure. A four-minute recovery period can be allowed between each of the stimulations or passive lengthening, with muscle length being mainlined at L₀. Procedure (a) can generate a higher peak stress compared to procedures (b)-(c). Procedure (b) can generate a moderate peak stress compared to rest of the procedures while procedure (c) can generate a low peak stress with no activation. In procedures (a) and (c), the muscles can be lengthened by about 10-20% of the original fiber length (L₀). Isometric force can be measured for each contraction just before the onset of the stretch. Force drop between the first and the last contraction can be correlated with the muscle membrane damage. A larger force drop can be correlated with greater muscle membrane damage. The percentage force drop can be calculated using the equation: Force drop=100 (force at the first contraction-force at the last contraction)/force at the first contraction). The test compound with smaller or less acute force drop in treated mdx muscles compared to untreated mdx or control muscles can further be evaluated.

### Example 2: Ex Vivo Assay for Assessing Muscle Membrane Integrity

Muscles from control and mdx mice can be prepared and can be subjected to the procedures described in Example 1 to assess the efficacy of a test compound in maintaining muscle membrane integrity. The treated and untreated control as well as mdx muscles can both be submerged in the oxygenated 0.2% procion orange/Ringer's solution for a total duration of 90 min. An internal control comprising non-stimulated contralateral counterpart can be also be used and submerged in the solution. The muscles can then be rinsed in normal Ringer's solution for 5 min twice then snap frozen, mounted and sectioned for histology. Muscle fibers with damaged membranes can take up the dye and can be scored as dye-positive fibers. Muscle fibers with intact membranes cannot take up the dye and can be scored as dye-negative fibers. Membrane integrity of the muscles can be assessed by determining the percentage of dye-positive fibers using fluorescence microscopy. Edges of the muscle sections can be excluded from scoring to avoid fibers potentially damaged due to muscle dissection or sectioning artifact. The test compound with higher percentage of dye-negative fibers in mdx muscles compared to untreated mdx, control muscles, or internal control can further be evaluated.

### Example 3: In Vivo Assay for Assessing Activities of Daily Living (ADL) or Habitual Physical Activities

ADL assessment can be used for determining muscle strength in control and dystrophic subjects before and after administering the test compound. ADL comprises self-care tasks that include, but are not limited to: Bathing and showering, personal hygiene and grooming (including brushing/combing/styling hair), dressing, toilet hygiene (getting to the toilet, cleaning oneself, and getting back up), functional mobility, and self-feeding (not including cooking or chewing and swallowing). Functional mobility may also be referred to as “transferring”, as measured by the ability to walk, get in and out of bed, and get into and out of a chair. A test compound can be administered to control and dystrophic individuals for assessing efficacy of the test compound in carrying out ADL. The test compound resulting in improved ADL in dystrophic subjects compared with pretreatment condition or with control subjects can further be evaluated.

### Example 4: In Vivo Assay for Assessing Muscle Strength

Voluntary assays, such as grip strength and leg press, can be used to assess muscle strength in control and dystrophic subjects before and after administering the test compound. Hand grip strength can be quantified by measuring the amount of static force that the hand can squeeze around a dynamometer. The force most commonly is measured in kilograms and pounds, but also in milliliters of mercury and in Newton. Hand dynamometers, such as Jamar, Dexter and Baseline, can be used. In some cases, the test compound resulting in improved grip strength in dystrophic subjects compared with pretreatment condition or with control subjects can further be evaluated.

The leg press can be a diagonal or vertical “sled” leg press or “cable” type leg press, or “seated leg press” type leg press. Weight disks (plates) are attached directly to the sled, which is mounted on rails. The user sits below the sled and pushes it upward with their feet. These machines normally include adjustable safety brackets that prevent the user from being trapped under the weight. The user sits upright and pushes forward with their feet onto a plate that is attached to the weight stack by means of a long steel cable.

Involuntary assays, such as isolated limb assay, can be used to assess the muscle strength in control and dystrophic subjects before and after administering the test compound. The pharmacodynamics response to the test compound can be determined by measuring the force-frequency relationship of tibialis anterior muscle contraction elicited by transcutaneous electrical stimulation of the deep fibular nerve. To measure tibialis anterior muscle force, adjustable, rigid chair frames with integrated footplates incorporating a force sensor can be used. Each subject can be fitted into the chair, and the right foot can be strapped firmly to the footplate with the lower leg and knee immobilized. The chairs can be constructed so that, when seated, the subject's knees are bent approximately 60, and the ankle angle is fixed at 105 (shin to bottom of foot). A strain-gauge containing a load cell (MLP-75; Transducer Techniques, Temecula, California) coupled to the bottom of the foot-plate can be used to measure dorsiflexion force. An adhesive surface electrode (61-2510; ConMed, USA) fixed to the lateral aspect of the upper leg just below the head of the fibula can be used as the cathode and delivered stimulation pulses transcutaneously to the deep fibular nerve. The anode can be placed on the medial aspect of the knee. To identify optimal cathode placement, a hand-held, non-adhesive electrode through which low-intensity stimulation pulses can be delivered is used to activate the nerve without stimulating antagonistic muscle groups, as determined by palpation. The stimulus intensity can be set by slowly increasing the electrical current during each stimulation pulse until the magnitude of the tibialis anterior twitch force and the resulting electromyogram (EMG) signal do not increase in magnitude. The final stimulus current can then be set approximately 20% greater to ensure maximal nerve activation throughout the dosing period. The force-frequency response of each subject can be measured at baseline, and at 1, 3, 5, and 7 hours post-dose during each of the 4 dosing periods in control and dystrophic subjects. Each stimulation protocol can consist of 5-, 7.5-, 10-, 12.5-, 15-, 17.5-, 25-, and 50-HZ stimulation trains of 0.5-ms pulse width and 800-ms duration. The stimulation frequency can be delivered in random order so subjects could not anticipate the intensity of the stimulus with a single stimulus pulse delivered 5 s before and 5 s after each stimulus train to elicit a twitch response. Twitch-train-twitch sequences can be separated by 30 s. At each assessment time-point, the stimulation protocol can be performed in triplicate, and commensurate blood samples can be taken to measure the test compound plasma concentrations. The data acquisition system can be used to create stimulation pulse trains, amplify the EMG, and measure the strain-gauge output can be custom designed. The test compound resulting in decreased force frequency response in dystrophic subjects compared with pretreatment condition or with control subjects can further be evaluated. A test compound may suppress high frequency force generation in such an involuntary test system. Such assays may be used to establish drug pharmacokinetics and pharmacodynamics.

Other in vivo assays can include activity monitors, heart monitors, etc.

### Example 5: In Vivo Assay Using Blood Biomarkers

Blood biomarkers can be used to assess efficacy of the test compound in control and dystrophic subjects before and after administering the test compound. Serum creatine kinase (CK) levels can be correlated with the extent of muscle damage. CK levels can be determined by a Hitachi Modular PT automated clinical chemistry analyser (Roche, Germany) with a commercially available instrument. The test compound resulting in reduction in CK levels in dystrophic subjects compared with pretreatment value can further be evaluated.

CK levels can also be correlated with troponin (TnI) levels. In addition to or in place of CK levels, serum fast (fsTnI) and slow skeletal troponin I isoform (ssTnI) concentrations can be determined. TnI levels can be determined by using enzyme-linked immunosorbent assays. The test compound resulting in reduction in fsTnI levels in dystrophic subjects compared with control subjects can further be evaluated.

Muscle damage can induce an inflammatory response, causing inflammatory molecules to be released in the plasma. Levels of such inflammatory molecules can be used as biomarkers for determining muscle damage. Cytokines, such as TNFα, IL-1, IL-6 and IL-4, can be used as biomarkers of muscle damage by using immunosorbent assays, RT-PCR or microarrays. The test compound resulting in reduction in levels of inflammatory biomarkers in dystrophic subjects compared with control subjects can further be evaluated.

### Example 6: In Vivo Assay for Assessing Bioavailability of the Test Compound

Bioavailability can refer to the extent and rate at which the test compound enters systemic circulation, thereby accessing the site of action. Bioavailability can differ based on the method of administration. A test compound administered intravenously can have bioavailability of 100%. A test compound administered via other routes, such as oral, can have decreased bioavailability relative to intravenously administered test compound.

Bioavailability can be absolute or relative. Absolute bioavailability can be determined by comparing the bioavailability of the test compound in systemic circulation following non-intravenous administration, such as oral, ocular, rectal, transdermal, subcutaneous, or sublingual, with the bioavailability of the same test compound following intravenous administration. Relative bioavailability can be determined by measuring the bioavailability of a test compound when compared with another test compound.

Bioavailability can be assessed by determining concentration of the test compound in plasma (plasma concentration) over time after administering the test compound. Bioavailability can be measured by determining area under the plasma concentration-time curve (an AUC). Plasma concentration of the test compound can be correlated with extent of the absorption of the test compound. Plasma concentration of the test compound can increase with extent of absorption. The maximum (peak) plasma concentration can reach when drug elimination rate equals absorption rate. Peak time (when maximum plasma drug concentration occurs) can be used general index of absorption rate. The later peak time can be correlated with the slower the absorption.

Bioavailability can be estimated by measuring the total amount of test compound excreted after a single dose. Urine can be collected over a period of 7 to 10 elimination half-lives for complete urinary recovery of the absorbed test compound. After multiple dosing, bioavailability may be estimated by measuring unchanged drug recovered from urine over a 24-h period under steady-state conditions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

1. A method of treating a neuromuscular condition, comprising administering to a subject in need thereof an inhibitor of skeletal muscle contraction wherein the inhibitor of skeletal muscle contraction reduces skeletal muscle contraction by 5% to 90% in an ex vivo assay wherein:
a. extensor digitorum longus muscle dissected from a mouse is mounted on a lever of a servomotor system and the muscle is bathed in an oxygenated Krebs solution to maintain muscle function;
b. a test compound is applied to the muscle;
c. an isometric contraction step is performed wherein the muscle is stimulated with a series of six electrical pulses, or an eccentric contraction step is performed wherein the muscle is stimulated with a series of electrical pulses and stretched to 10% to 20% greater than its rested length, wherein following each pulse, the force generated by the muscle contraction is measured;
d. the change in force generated by the muscle contraction over the series of electrical pulses in step c. is calculated as the test force drop and compared to the change in force generated by the muscle contraction over the series of electrical pulses in a control sample without exposure to the test compound is calculated as the control force drop; and
e. the test force drop and control force drop are compared to measure skeletal muscle contraction.

2. The method of claim 1, wherein the neuromuscular condition is selected from Duchenne muscular dystrophies, Becker muscular dystrophy, myotonic dystrophy 1, myotonic dystrophy 2, facioscapulohumeral muscular dystrophy, oculopharyngeal muscular dystrophy, limb girdle muscular dystrophy, tendinitis, carpal tunnel syndrome.

3. The method of claim 2, wherein said neuromuscular condition is Duchenne muscular dystrophy.

4. The method of claim 2, wherein said neuromuscular condition is Becker muscular dystrophy.

5. The method of claim 2, wherein said neuromuscular condition limb girdle muscular dystrophy.

6. The method of claim 1, wherein the inhibitor of skeletal muscle contraction is selected from an inhibitor of myosin.

7. The method of claim 6, wherein the inhibitor of myosin is an inhibitor of skeletal muscle myosin II.

8. The method of claim 7, wherein the inhibitor of skeletal muscle myosin II is administered in an amount sufficient to reduce muscle contractions by 25% to 75%.

9. The method of claim 1, wherein the inhibitor of skeletal muscle contraction does not impact activities of daily living (ADL) or habitual physical activity.

10. The method of claim 1, wherein the method further comprises measuring skeletal muscle contraction or force from said skeletal muscle contraction of said subject prior to and following administration of said skeletal muscle myosin II inhibitor to said subject.

11. The method of claim 10, wherein said skeletal muscle contraction of said subject prior to said administering is within 20% of said skeletal muscle contraction following said administering to said subject.

12. The method of claim 11, wherein said skeletal muscle contraction of said subject prior to said administering is within 10% of said muscle contraction following said administering to said subject.

13. The method of claim 7, wherein the inhibitor of skeletal muscle myosin II inhibits ATPase activity but does not inhibit cardiac muscle myosin S1 ATPase in vitro assays.

14. The method of claim 7, wherein the inhibitor of skeletal muscle myosin II does not appreciably inhibit cardiac muscle contraction or force from said cardiac muscle contraction of said subject.

15. (canceled)

16. The method of claim 7, wherein the inhibitor of skeletal muscle myosin II does not appreciably inhibit smooth muscle contraction.

17. The method of claim 1, wherein the inhibitor of skeletal muscle contraction is a sulfonamide, a hydroxycoumarin, a pyridazinone, or a pyrrolidinone.

18. The method of claim 17, wherein the inhibitor of skeletal muscle contraction is a sulfonamide.

19. The method of claim 18, wherein the inhibitor of skeletal muscle contraction is an optionally substituted N-benzyl-p-tolyl-sulfonamide.

20. The method of claim 17, wherein the inhibitor of skeletal muscle contraction is a pyridazinone.

21. A method of treating a neuromuscular condition, comprising administering to a subject in need thereof an inhibitor of skeletal muscle contraction, wherein the inhibitor of skeletal muscle contraction is administered in an amount that modulates creatine kinase by 5 to 90% relative to a pre-treatment creatine kinase level of said subject.
